(19) United States
(12) Patent Application Publication
(10) Pub. No.: US 2025/0127470 A1
(43) Pub. Date: Apr. 24, 2025

(54) CONTRAST ENHANCED BREAST IMAGING SYSTEMS AND METHODS

(71) Applicant: GE Precision Healthcare LLC, Waukesha, WI (US)

(72) Inventors: Clement Jailin, Buc (FR); Pablo Milioni De Carvalho, Chaville (FR); Laurence Vancamberg, Buc (FR)

(21) Appl. No.: 18/493,706

(22) Filed: Oct. 24, 2023

(51) Int. Cl. A61B 6/00; G06T 7/11

(52) U.S. Cl. CPC A61B 6/481; A61B 6/4258; A61B 6/502; A61B 6/5229; G06T 7/11; G06T 2207/30068

(57) ABSTRACT

Methods and systems are provided for contrast enhanced breast imaging. A method includes automatically distinguishing a first region of interest (ROI) corresponding to breast parenchymal enhancement (BPE) in a contrast enhanced breast image. The method further includes automatically adjusting an appearance of the first ROI, generating an adjusted BPE image, and displaying the adjusted BPE image and the contrast enhanced breast image at a display.

### TECHNICAL FIELD

Embodiments of the subject matter disclosed herein relate to systems and methods for contrast enhanced breast imaging.

### BACKGROUND

Contrast enhanced imaging is a screening/diagnostic method that may be used to visualize lesions in tissue, such as breast tissue. A contrast enhancing agent is administered intravenously and may localize in lesion tissue due to rapid neovascularization around the lesion causing imperfect blood vessels which may leak the contrast agent to the surrounding tissue. The contrast enhancement is particularly useful in imaging lesions in breasts having dense breast tissue. The contrast enhancing agent may additionally be delivered to normal, healthy breast tissue and show up in a contrast enhanced image as an enhanced contrast area in a phenomena called breast background parenchymal enhancement.

### BRIEF DESCRIPTION

In one embodiment, a method includes automatically distinguishing a first region of interest (ROI) of a contrast enhanced breast image, wherein the first ROI corresponds to background parenchymal enhancement (BPE), automatically adjusting an appearance of the first ROI and generating an adjusted BPE image, and displaying the contrast enhanced breast image and the adjusted BPE image at a display.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present invention will be better understood from reading the following description of non-limiting embodiments, with reference to the attached drawings, herein below:

FIG. 1 is a schematic illustration of a digital mammography system according to an embodiment;

FIG. 2 is a contrast enhanced mammography (CEM) image showing background parenchymal enhancement (BPE);

FIG. 3 is a flow chart of a method for displaying BPE free images according to an embodiment;

FIG. 4 is a CEM image showing a lesion and BPE;

FIG. 5 is the image of FIG. 4 without BPE;

FIG. 6 is a first example of display showing low energy, recombined, and BPE free images;

FIG. 7 is a second example of displays showing low energy, recombined, and BPE free images;

FIG. 8. is a third example of displays showing low energy, recombined, and BPE free images;

FIG. 9 is block diagram of an exemplary embodiment of an image processing system; and

FIG. 10 is a block diagram of an exemplary embodiment of methods for identifying and displaying BPE and suspicious areas of contrast enhanced breast images.

### DETAILED DESCRIPTION

The following description relates to systems and methods for displaying contrast enhanced breast images. The contrast enhanced breast images may be contrast enhanced digital mammography (CEM) images or contrast enhanced breast magnetic resonance imaging (MRI) images or contrast enhanced breast computed tomography (CT). In both CEM and contrast enhanced breast MRI and contrast enhanced breast CT a contrast agent is administered intravenously and preferentially localizes in lesions or other suspicious areas due to leaky neovascularized blood vessels surrounding the lesion. Background parenchymal enhancement (BPE) may also occur during CEM or contrast enhanced MRI procedures or contrast enhanced CT procedures, wherein contrast agent also localizes in normal, healthy breast tissue. Clinicians reviewing CEM or contrast enhanced breast MRI or contrast enhanced breast CT images may seek to differentiate a lesion from BPE. Methods and systems for automatically adjusting contrast BPE in contrast enhanced breast images may aid the clinician in quickly and accurately identifying whether or not a lesion is present in the contrast enhanced breast image in addition to accurately defining a location of the lesion within the image if present. Alternatively, a presence or absence of BPE may also be information used by a clinician to aid in diagnosis and cancer risk estimation and increased contrast of the BPE may be desired.

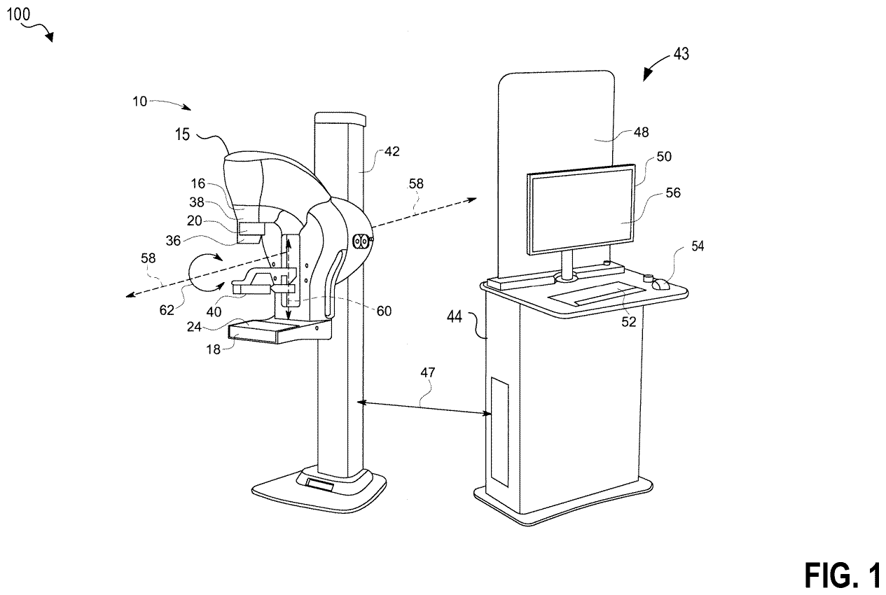

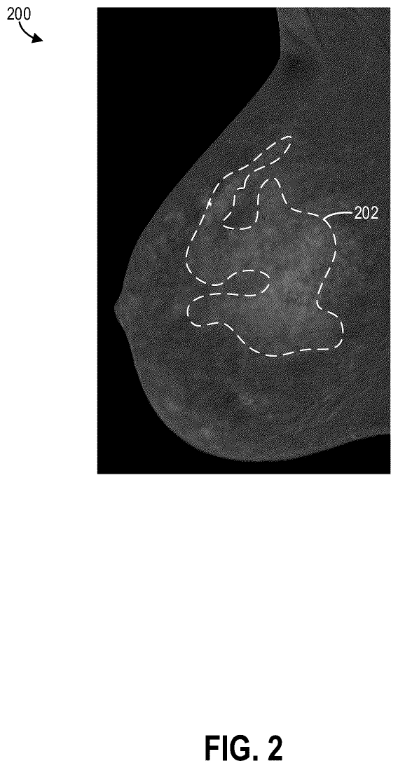

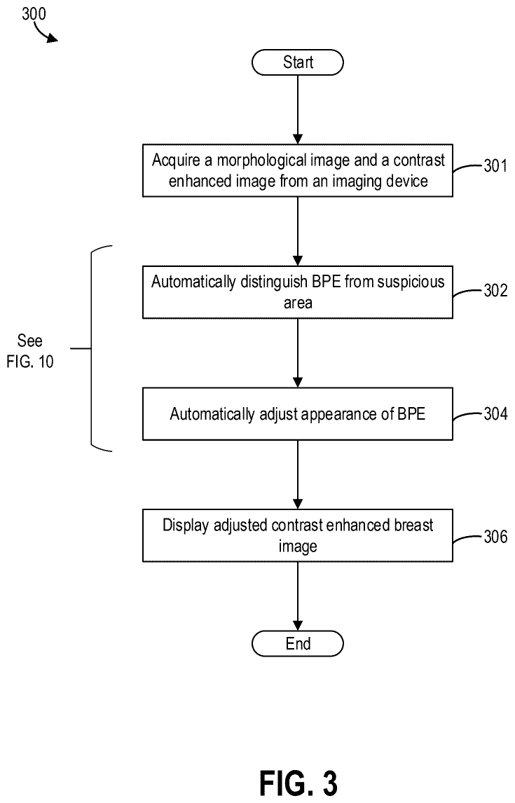

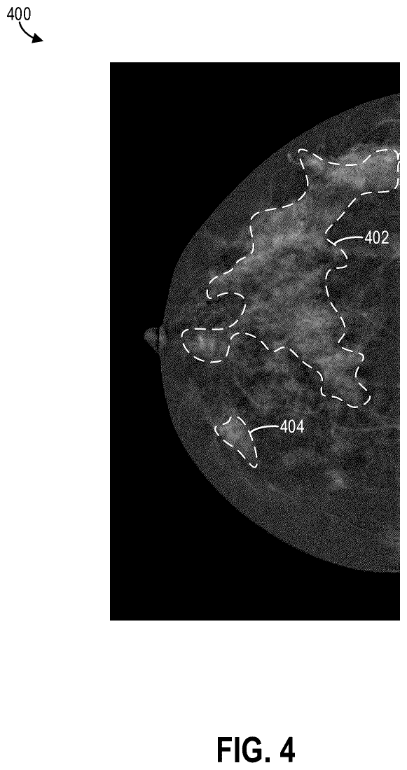

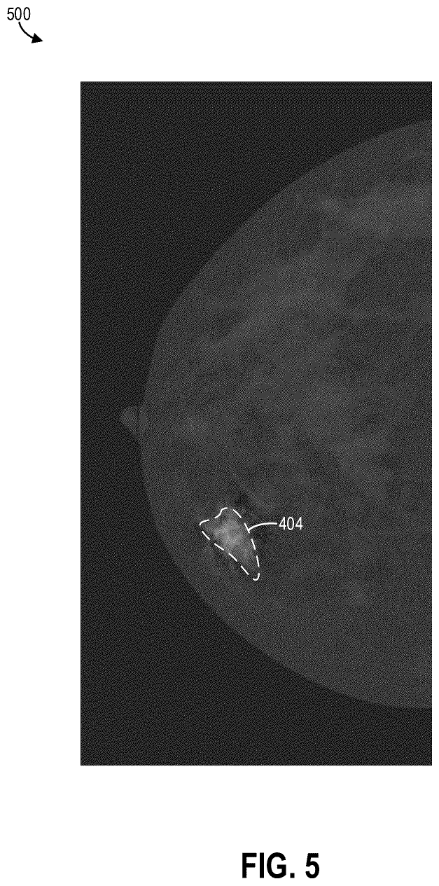

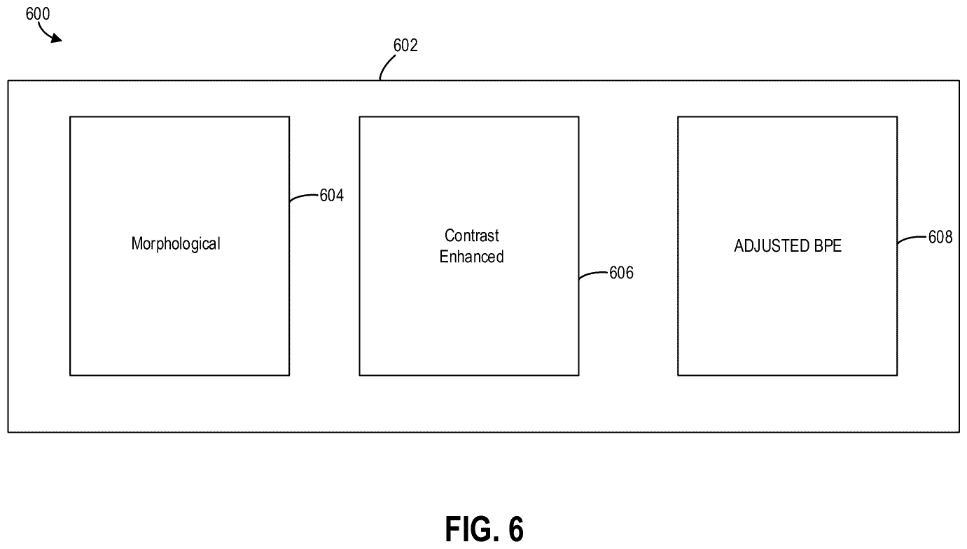

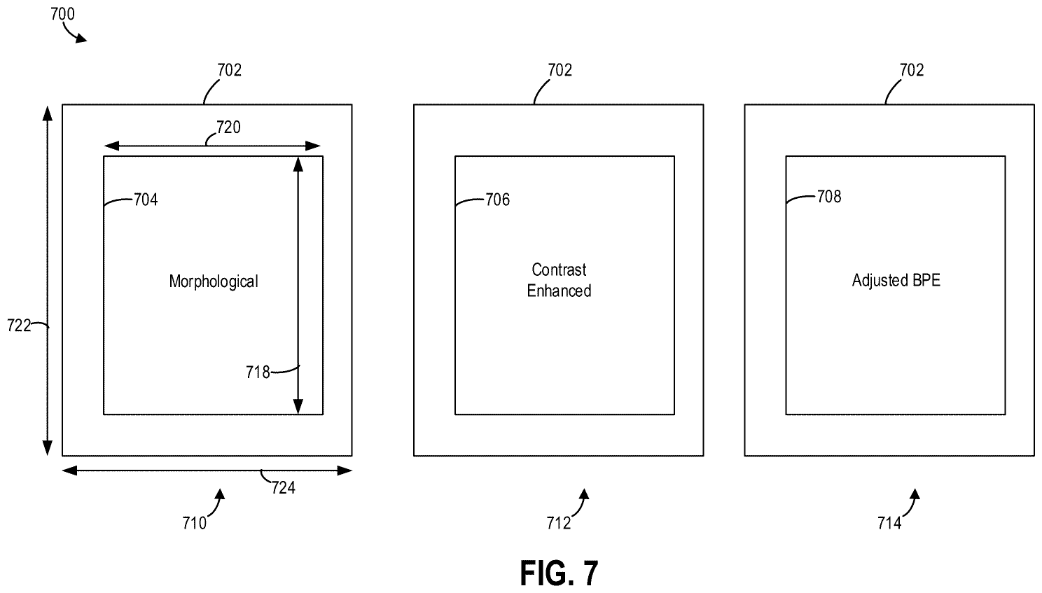

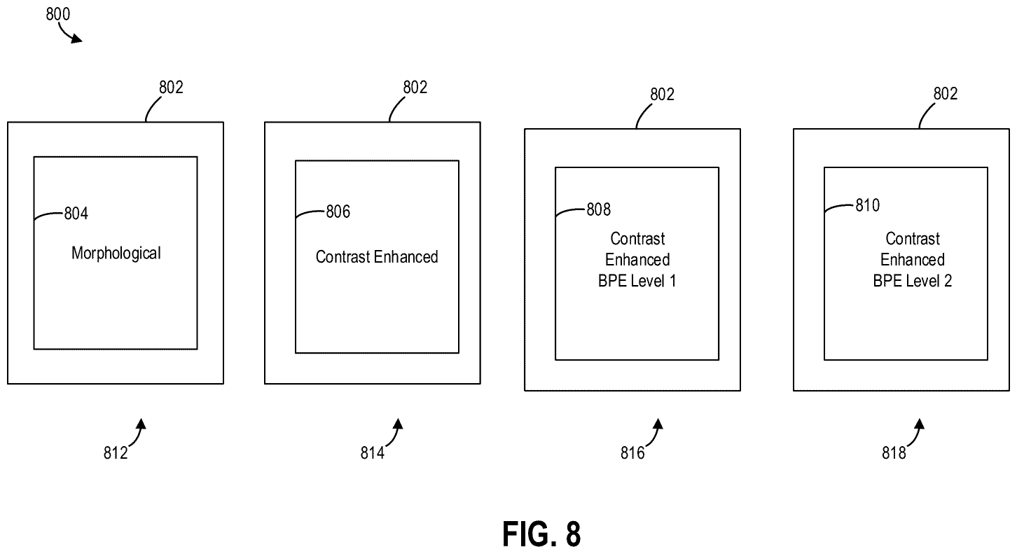

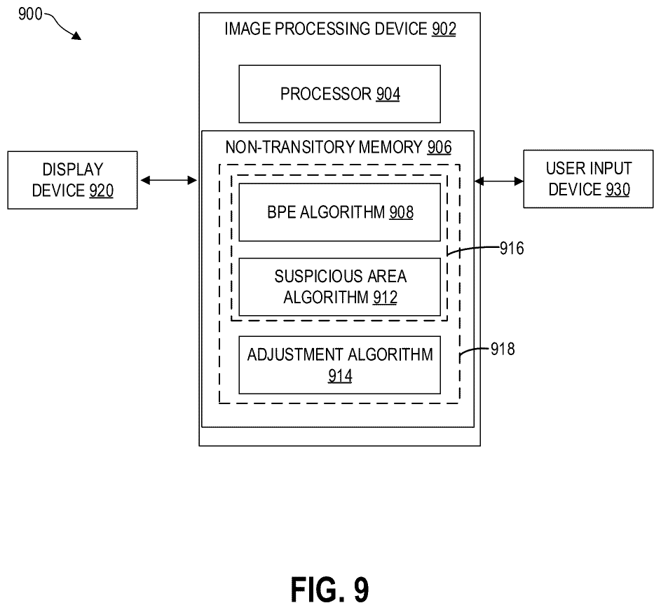

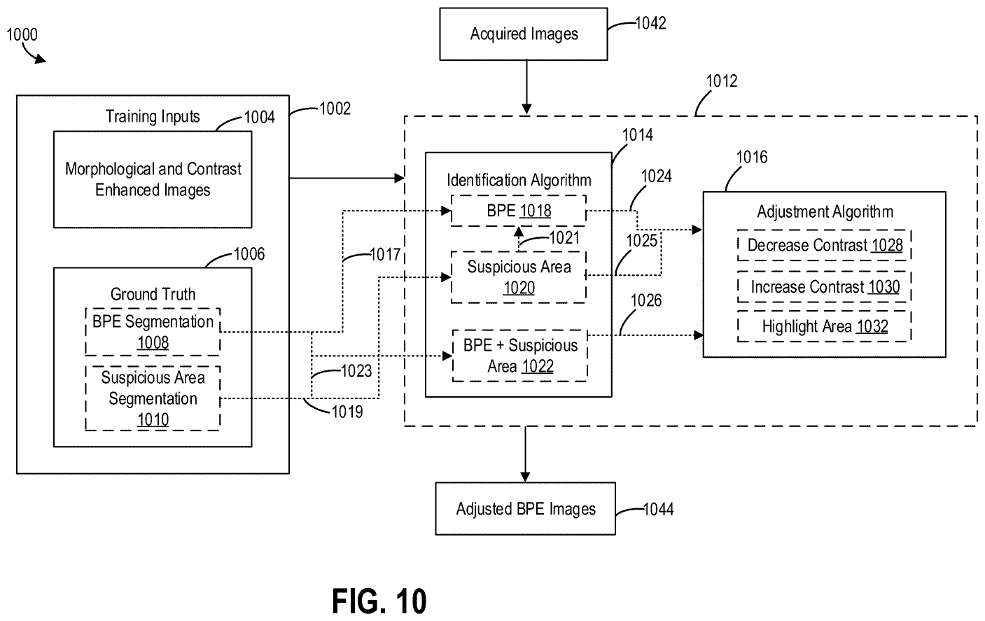

A block diagram of an imaging processing system which may store one or more methods for identifying and displaying CEM and/or contrast enhanced breast MRI images and/or contrast enhanced breast CT is shown in FIG. 9. The image processing system may be communicatively coupled to an imaging system which may be an MRI system or a digital mammography system or a breast CT system. A schematic of a digital mammography system is shown in FIG. 1. The digital mammography system may be used to take CEM images which may be automatically processed and displayed by a method for automatically differentiating BPE from a suspicious area and adjusting the BPE displayed in the image. The BPE adjusting and display system and method may also be applied to any contrast enhanced breast image, such as those obtained by contrast enhanced breast MRI or by contrast enhanced breast CT. An example of a CEM image including BPE is shown in FIG. 2. The BPE may cause difficulties for a clinician to properly distinguish any lesions, if present, from healthy tissue. The clinician may use a method such as the method shown in FIG. 3 to automatically adjust BPE of a contrast enhanced breast image. As a first step, the method may automatically distinguish BPE contrast enhancement from a suspicious area contrast enhancement. An example of a CEM image with regions of interest (ROIs) distinguished as either BPE or a suspicious area is shown in FIG. 4. After the lesion and BPE regions of the contrast enhanced image are distinguished and the image is adjusted, a BPE free image may be displayed to the clinician. The image of FIG. 4 is shown in FIG. 5 with contrast of the BPE region adjusted to form a BPE free image. Herein, a BPE free image may refer to an image where contrast in areas determined to be BPE are adjusted to background levels. Algorithms may be used to distinguish BPE and suspicious area and to adjust a display of BPE in the image. FIG. 10 shows an example of a block diagram for using algorithms which may execute some of the steps of the method shown in FIG. 3. The method of FIG. 3 may output images for a clinician to review. When reviewing images, the clinician may review a low energy image (for CEM) or other image in which the contrast media does not affect a contrast of the image, a recombined image showing all of the contrast enhancement in addition to a BPE free and/or reduced BPE image. Depending on preferences of the clinician, a display size, and/or a complexity of the images, among other factors, multiple display options may be desired. As a first example, the low energy, recombined, and BPE free image may be displayed side by side as shown in FIG. 6. As a second example, the clinician may view a single image at a time which gradually transitions between the low energy, recombined, and BPE free images as shown in FIG. 7. As a third example, the clinician may scroll between a low energy image, recombined image and images with adjusted BPE at different levels as shown in FIG. 8.

Turning first to FIG. 9, an image processing system 900 is shown, in accordance with an exemplary embodiment. In some embodiments, image processing system 900 is incorporated into an imaging system, such as a medical imaging system. In some embodiments, at least a portion of image processing system 900 is disposed at a device (e.g., edge device, server, etc.) communicably coupled to a medical imaging system via wired and/or wireless connections. In some embodiments, at least a portion of the image processing system 900 is disposed at a device (e.g., a workstation), located remote from a medical imaging system, which is configured to receive images from the medical imaging system or from a storage device configured to store images acquired by the medical imaging system. Image processing system 900 may comprise image processing device 902, user input device 930, and display device 920. In some embodiments, image processing device 902 may be communicably coupled to a picture archiving and communication system (PACS), and may receive images from, and/or send images to, the PACS.

Image processing device 902 includes a processor 904 configured to execute machine readable instructions stored in non-transitory memory 906. Processor 904 may be single core or multi-core, and the programs executed thereon may be configured for parallel or distributed processing. In some embodiments, the processor 904 may optionally include individual components that are distributed throughout two or more devices, which may be remotely located and/or configured for coordinated processing. In some embodiments, one or more aspects of the processor 904 may be virtualized and executed by remotely-accessible networked computing devices configured in a cloud computing configuration.

Non-transitory memory 906 may store BPE algorithm 908, suspicious area algorithm 912, and adjustment algorithm 914 which may be used in executing the method for displaying contrast enhanced breast images described below with respect to FIG. 3. In some examples, BPE algorithm 908 and suspicious area algorithm 912 may be combined into a single BPE and suspicious area algorithm 916. In alternate embodiments, BPE algorithm 908, suspicious area algorithm 912, and adjustment algorithm 914 may be combined into a single identification/adjustment algorithm 918. Details of BPE algorithm 908, suspicious area algorithm 912, and adjustment algorithm 914 may be discussed further below with respect to FIG. 10.

In some embodiments, the non-transitory memory 906 may include components disposed at two or more devices, which may be remotely located and/or configured for coordinated processing. In some embodiments, one or more aspects of the non-transitory memory 906 may include remotely-accessible networked storage devices configured in a cloud computing configuration.

Image processing system 900 may further include user input device 930. User input device 930 may comprise one or more of a touchscreen, a keyboard, a mouse, a trackpad, a motion sensing camera, or other device configured to enable a user to interact with and manipulate data within image processing system 900. In some embodiments, user input device 930 enables a user to select one or more types of ROI to be segmented in a medical image.

Display device 920 may include one or more display devices utilizing virtually any type of technology. In some embodiments, display device 920 may comprise a computer monitor, a touchscreen, a projector, or other display device known in the art. Display device 920 may be configured to receive data from image processing device 902, and to display a contrast enhanced breast image, modified to hide or highlight BPE and suspicious areas of the image. In some embodiments, image processing device 902 may determine a standard view classification of a medical image, may select a graphical user interface (GUI) based on the standard view classification of the image, and may display via display device 920 the medical image and the GUI. Display device 920 may be combined with processor 904, non-transitory memory 906, and/or user input device 930 in a shared enclosure, or may be a peripheral display device and may comprise a monitor, touchscreen, projector, or other display device known in the art, which may enable a user to view images, and/or interact with various data stored in non-transitory memory 906.

It may be understood that image processing system 900 shown in FIG. 9 is for illustration, not for limitation. Another appropriate image processing system may include more, fewer, or different components.

Turning now to FIG. 1, a digital mammography system 100 including an x-ray system 10 for performing a mammography procedure is shown, according to an embodiment of the disclosure. Digital mammography system 100 may be an example of an imaging processing device, such as image processing device 902 described above with respect to FIG. 9. In some examples, the x-ray system 10 may be a tomosynthesis system, such as a digital breast tomosynthesis (DBT) system. Further, the x-ray system 10 may be used to perform one or more procedures including digital tomosynthesis imaging, and DBT guided breast biopsy.

The x-ray system 10 includes a support structure 42, to which a radiation source 16, a radiation detector 18, and a collimator 20 are attached. The radiation source 16 is housed within a gantry 15 that is movably coupled to the support structure 42. In particular, the gantry 15 may be mounted to the support structure 42 such that the gantry 15 including the radiation source 16 can rotate around an axis 58 in relation to the radiation detector 18. An angular range of rotation of the gantry 15 housing the radiation source 16 indicates a rotation up to a desired degree in either direction about the axis 58. For example, the angular range of rotation of the radiation source 16 may be −θ to +θ, where θ may be such that the angular range is a limited angle range, less than 360 degrees. An exemplary x-ray system may have an angular range of ±11 degrees, which may allow rotation of the gantry (that is rotation of the radiation source) from −11 degrees to +11 degrees about an axis of rotation of the gantry. The angular range may vary depending on the manufacturing specifications. For example, the angular range for DBT systems may be approximately ±11 degrees to ±60 degrees, depending on the manufacturing specifications.

The radiation source 16 is directed toward a volume or object to be imaged and is configured to emit radiation rays at desired times to acquire one or more images. The radiation detector 18 is configured to receive the radiation rays via a surface 24. The detector 18 may be any one of a variety of different detectors, such as an x-ray detector, digital radiography detector, or flat panel detector. The collimator 20 is disposed adjacent to the radiation source 16 and is configured to adjust an irradiated zone of a subject.

In some embodiments, the system 10 may further include a patient shield 36 mounted to the radiation source 16 via face shield rails 38 such that a patient's body part (e.g., head) is not directly under the radiation. The system 10 may further include a compression paddle 40, which may be movable upward and downward in relation to the support structure along a vertical axis 60. Thus, the compression paddle 40 may be adjusted to be positioned closer to the radiation detector 18 by moving the compression paddle 40 downward toward the detector 18, and a distance between the detector 18 and the compression paddle 40 may be increased by moving the compression paddle upward along the vertical axis 60 away from the detector. The movement of the compression paddle 40 may be adjusted by a user via compression paddle actuator (not shown) included in the x-ray system 10. The compression paddle 40 may hold a body part, such as a breast, in place against the surface 24 of the radiation detector 18. The compression paddle 40 may compress the body part and hold the body part still in place while optionally providing apertures to allow for insertion of a biopsy needle, such as a core needle or a vacuum assisted core needle. In this way, compression paddle 40 may be utilized to compress the body part to minimize the thickness traversed by the x-rays and to help reduce movement of the body part due to the patient moving. The x-ray system 10 may also include an object support (not shown) on which the body part may be positioned.

The digital mammography system 100 may further include workstation 43 comprising a controller 44 including at least one processor and a memory. The controller 44 may be communicatively coupled to one or more components of the x-ray system 10 including one or more of the radiation source 16, radiation detector 18, the compression paddle 40, and a biopsy device. In an embodiment, the communication between the controller and the x-ray system 10 may be via a wireless communication system. In other embodiments, the controller 44 may be in electrical communication with the one or more components of the x-ray system via a cable 47. Further, in an exemplary embodiment, as shown in FIG. 1, the controller 44 is integrated into workstation 43. In other embodiments, the controller 44 may be integrated into one or more of the various components of the system 10 disclosed above. Further, the controller 44 may include processing circuitry that executes stored program logic and may be any one of different computers, processors, controllers, or combination thereof that are available for and compatible with the various types of equipment and devices used in the x-ray system 10.

The workstation 43 may include a radiation shield 48 that protects an operator of the system 10 from the radiation rays emitted by the radiation source 16. The workstation 43 may further include a display 50, a keyboard 52, mouse 54, and/or other appropriate user input devices that facilitate control of the system 10 via a user interface 56.

Controller 44 may adjust the operation and function of the x-ray system 10. As an example, the controller 44 may provide timing control, as to when the x-ray source 16 emits x-rays, and may further adjust how the detector 18 reads and conveys information or signals after the x-rays hit the detector 18, and how the x-ray source 16 and the detector 18 move relative to one another and relative to the body part being imaged. The controller 44 may also control how information, including images 42 and data acquired during the operation, is processed, displayed, stored, and manipulated. Various processing steps as described herein with respect to FIGS. 2 and 3, performed by the controller 44, may be provided by a set of instructions stored in non-transitory memory of controller 44.

Further, as stated above, the radiation detector 18 receives the radiation rays emitted by the radiation source 16. In particular, during imaging with the x-ray system, a projection image of the imaged body part may be obtained at the detector 18. In some embodiments, data, such as projection image data, received by the radiation detector 18 may be electrically and/or wirelessly communicated to the controller 44 from the radiation detector 18. The controller 44 may then reconstruct one or more scan images based on the projection image data, by implementing a reconstruction algorithm, for example. The reconstructed image may be displayed to the user on the user interface 56 via the display 50 (e.g., a screen).

The radiation source 16, along with the radiation detector 18, forms part of the x-ray system 10 which provides x-ray imagery for the purpose of one or more of screening for abnormalities, diagnosis, dynamic imaging, and image-guided biopsy. For example, the x-ray system 10 may be operated in a mammography mode for screening for abnormalities. During mammography, a patient's breast is positioned and compressed between the detector 18 and the compression paddle 40. Thus, a volume of the x-ray system 10 between the compression paddle 40 and the detector 18 is an imaging volume. The radiation source 16 then emits radiation rays onto the compressed breast, and a projection image of the breast is formed on the detector 18. The projection image may then be reconstructed by the controller 44, and displayed on the interface 50. During mammography, the gantry 15 may be adjusted at different angles to obtain images at different orientations, such as a cranio-caudal (CC) image and a medio-lateral oblique (MLO) image. In one example, the gantry 15 may be rotated about the axis 58 while the compression paddle 40 and the detector 18 remain stationary. In other examples, the gantry 15, the compression paddle 40, and the detector 18 may be rotated as a single unit about the axis 58.

In some examples, breast imaging systems such as digital mammography system 100 may be configured to perform contrast imaging where contrast agents, such as iodine, can be injected into the patient that travel to the region of interest (ROI) within the breast (e.g., a lesion). The contrast agents are taken up in the blood vessels surrounding a cancerous lesion in the ROI, thereby providing a contrasting image for a period of time with respect to the surrounding tissue, enhancing the ability to locate the lesion. In some examples, the contrast agent may additionally be taken up by healthy breast tissue causing BPE to be visible in the acquired image.

The use of a contrast agent can be coupled with images of the ROI taken using dual-energy imaging processes and technology. In dual-energy imaging, low-energy (LE) and high-energy (HE) images are taken of the ROI. For each view, a pair of images is acquired: a low-energy (LE) image and a high-energy (HE) image. The LE and HE images are usually obtained at mean energies above and below the k-edge of the contrast agent. At x-ray energies just above the k-edge of the contrast agent, the absorption of x-rays is increased resulting in an increase of contrast from the iodine contrast agent in the HE image.

In dual-energy 3D or stereotactic procedures, LE and HE image acquisitions are performed, with at least two different positions of the x-ray source with respect to the detector. The images are then recombined to display material-specific information with regard to the internal structure of the tissue being imaged. In examples where contrast enhanced breast MRI is used, the contrast agent may have a different Ti that may be exploited by a pulse sequence of the MRI. In either the MRI or CEM examples, contrast agent may pool in healthy breast tissue causing BPE.

An example of a recombined CEM image 200 of a breast is shown in FIG. 2. The breast in image 200 does not include a lesion or other type of suspicious area, however there is still region 202 which is shown as increased contrast (e.g. lighter) than surrounding breast tissue. Region 202 may be due to BPE. BPE may be healthy tissue, but levels of BPE may be linked to risk for developing cancerous lesion. For this reason, a clinician may also be interested in identifying BPE in a CEM or other contrast enhanced image in addition to or instead of identifying a suspicious area.

Turning now to FIG. 3, a flow chart of a method 300 for displaying BPE free images is shown. Method 300 may be executed using computer readable instructions stored in the non-transitory memory of a computing device of an imaging system (e.g., imaging processing device 902 of FIG. 9 and/or digital mammography system 100 of FIG. 1) or a controller communicatively coupled to the digital mammography system (e.g., controller 44 of FIG. 1). In alternate embodiments, the controller may be included in or communicatively coupled to a MRI system. In some embodiments, method 300 may be executed by another computing device without departing from the scope of this disclosure (e.g., an edge device, a picture archiving and communication system (PACS)).

At 301, method 300 includes acquiring a morphological image and a contrast enhanced image from an imaging device. The morphological image may correspond to an image in which the contrast agent is not captured in the image and the contrast enhanced image may correspond to an image in which the contrast agent is captured in the image. In some examples, the imaging device may be a digital mammography system, such as digital mammography system 100 of FIG. 1 and the morphological image may be a lower energy x-ray image and the contrast enhanced image may result from a recombination of the low energy x-ray image (e.g., low energy image) and a high energy x-ray image. In alternate examples, the imaging device may be an MRI or a CT. Acquiring a morphological image and a contrast enhanced image may include, before acquiring the images, intravenous administration of a contrast agent followed by image capture. A processing device, such as a controller of the imaging device or a processor of an imaging system, may include instructions to generate a recombined image from the low energy image and the high energy image. The recombination instructions may include to do a logarithmic weighted subtraction between the low energy image and the high energy image. The contrast enhanced image may include regions of interests where contrast media has accumulated and shown up as areas of increased contrast compared to a background contrast of the recombined image. The morphological image and contrast enhanced image may be two-dimensional or three-dimensional image.

At 302, method 300 includes automatically distinguishing a BPE region from a suspicious area. Automatically distinguishing the BPE region from the suspicious area may be triggered by a user request to view the contrast enhanced image. The user request may be received by a user input device, such as user input device 930 of image processing device 902 or a user input device of workstation 43 of digital mammography system 100. In some examples, the suspicious area may be a lesion. In alternate examples, the suspicious area may be scars, metallic objects, and/or implants. Distinguishing the BPE region from the suspicious area may include identifying a region of interest (ROI) of the image and assigning the ROI as corresponding to BPE or corresponding to the suspicious area. Distinguishing BPE regions from suspicious areas may be performed automatically by algorithms stored in non-volatile memory of the controller. The algorithms may include a first algorithm configured to identify BPE and a second algorithm configured to identify the suspicious area. In some examples, a third algorithm may be configured to identify both the suspicious area and the BPE. Details of the algorithms may be explained further with respect to the block diagram of FIG. 10.

Turning to FIG. 4, an example of a recombined CEM image 400 is shown. Image 400 includes both BPE and a lesion (e.g., suspicious area). A first region of interest 402 and a second region of interest 404 may be segmented in image 400. The first ROI and second ROI may be automatically segmented by an algorithm including machine learning algorithms and/or deep learning algorithms, or may be segmented by a clinician. In some examples the algorithms configured to identify BPE and the suspicious area may also include instructions to automatically segment regions of interest. A contrast of each of the first ROI and the second ROI may be above a threshold level above a background contrast level. First ROI 402 may correspond to BPE while second ROI 404 may correspond to a lesion. First ROI 402 may be automatically distinguished from second ROI 404 by an algorithm as described further below with respect to FIG. 10. A clinician viewing image 400 may spend an increased amount of time to diagnose the patient or demand repeat or alternate imaging procedures if first ROI and second ROI are not automatically distinguished as BPE and the suspicious area.

Returning now to FIG. 3, method 300 proceeds to 304 and includes automatically adjusting an appearance of the BPE. Automatically adjusting the appearance may be performed by an adjustment algorithm stored in a memory of the controller. Automatically adjusting may occur after automatically distinguishing BPE from the suspicious area without any additional triggers. In some examples, along with a request for viewing a contrast enhanced image, the user may request a desired adjustment. For example, the user may request an increase, decrease, or highlighting of the BPE. In some examples, the adjustment algorithm may be combined with the algorithm that automatically distinguished BPE from the suspicious area. Algorithms for automatically distinguishing and adjusting BPE are discussed further below with respect to FIG. 10.

In a first embodiment, automatically adjusting the appearance of the BPE may include decreasing a contrast of the ROI corresponding to BPE to a level below the contrast level of the ROI corresponding to the suspicious area. In some examples, automatically reducing a contrast of the BPE may include generating a plurality of BPE reduced images, each image of the plurality of BPE reduced images may include contrast of the BPE reduced by a stepwise amount. In some examples the contrast level of the ROI corresponding to BPE may be reduced to a level equivalent to a background contrast level of the image, resulting in a BPE free image.

In a second embodiment, automatically adjusting the appearance of the BPE may include automatically increasing a contrast of the ROI corresponding to BPE. In some examples the contrast of the ROI corresponding to BPE may be increased to a level above a contrast of the ROI corresponding to the suspicious area. In further examples automatically increasing a contrast of the ROI corresponding to BPE may include generating a plurality of BPE increased images, each of the plurality of BPE increased imaged may include contrast of the BPE increased by a stepwise amount.

In a third embodiment, automatically adjusting the appearance of the BPE may include highlighting the ROI corresponding to BPE. Highlighting may include overlaying the ROI corresponding to BPE with a transparent colored overlay. In some examples the overlay may be a color that is different from the black, white, and greyscale colors of the contrast enhanced image.

Turning now to FIG. 5, an example of a recombined BPE free CEM image is shown. Image 500 may be the same as image 400, but without BPE (e.g., BPE free). An area of first ROI 402 of image 400 may no longer be distinguishable from background breast tissue in image 500. The second ROI 404 may still be identified in image 500 due to the contrast level of the second ROI 404 being maintained between image 400 and image 500. In this way, presence, location, and size of the suspicious area may be more easily identified by the clinician in image 500 than in image 400.

Returning now to FIG. 3, method 300 proceeds to 306 and includes displaying the adjusted contrast enhanced breast image. The display of BPE may be adjusted in the adjusted contrast enhanced breast image and may be herein referred to as an adjusted BPE image. Displaying the adjusted BPE image may include displaying the adjusted BPE image (e.g., adjusted contrast enhanced image) alongside a morphological image and a contrast enhanced image without BPE reduced. In examples where the image is acquired using digital mammography (e.g., contrast enhanced mammography), the morphological image may correspond to a low energy image and the contrast enhanced image may correspond to a recombined image. An arrangement type of images displayed may depend on, among other things, preference of the clinician, a complexity of the analysis, as well as an available screen area for displaying the images. In some examples, an arrangement type of images displayed may be automatically selected by the method based on a size of the display. For example, a first type of display may be automatically selected for a large format computer monitor display and a second type of display may be automatically selected for a small format computer monitor display. Examples of displaying the adjusted BPE image are discussed further below with respect to FIGS. 6-8. Method 300 Ends.

Turning now to FIG. 10 a block diagram 1000 exemplifying an embodiment of algorithms for automatically distinguishing BPE from a suspicious area and automatically adjusting an appearance of BPE is illustrated. The algorithms of FIG. 10 may be used in execution of steps 302 and 304 of method 300 as described above with respect to FIG. 3. The algorithms described below with respect to FIG. 10 may be stored as instructions in non-transitory memory of a controller. The algorithms may be stored in non-transitory memory of the same controller storing the instructions of method 300 or a different controller.

Training inputs 1002 may be training image pairs for an identification algorithm. A first part of the training pair 1004 may be an image of a breast where contrast is not highlighted (e.g., low energy image of a CEM procedure) and a corresponding contrast enhanced image (e.g., a recombined image of the CEM procedure). A second part of the training pair 1006 may be a ground truth, wherein the corresponding contrast enhanced image has been segmented. In some examples, the ground truth may be an image which is segmented by a clinician. In alternate examples, the ground truth may be provided by a segmentation algorithm and reviewed by a clinician. As one embodiment, the ground truth may include a BPE segmentation image 1008. The BPE segmentation image 1008 may include a contrast enhanced image where an ROI corresponding to BPE has been segmented and identified as BPE. As an alternate embodiment, the ground truth may include a suspicious area segmentation image 1010. The suspicious area segmentation image may include a contrast enhanced image where an ROI corresponding to a suspicious area, such as a lesion, has been segmented and identified. In some examples, the ground truth may include both BPE segmentation image 1008 and suspicious area segmentation image 1010. In examples where the ground truth includes both BPE segmentation image 1008 and suspicious area segmentation image 1010, they may be included in the same image and/or different images.

Training inputs 1002 may be used to train one or more algorithms. The one or more algorithms may include an identification algorithm 1014 and an adjustment algorithm 1016. Identification algorithm 1014 may be used in execution of step 302 of method 300 and adjustment algorithm 1016 may be used in execution of step 304 of method 300. In some examples, identification algorithm 1014 and adjustment algorithm 1016 may be combined into a single hybrid algorithm 1012 that is trained using both BPE segmentation image 1008 and suspicious area segmentation image 1010. As one example, single hybrid algorithm 1012 may be a machine learning/deep learning algorithm configured to transform images to highlight or erase BPE from a contrast enhanced breast image. Acquired images 1042 may be input into single hybrid algorithm 1012 and single hybrid algorithm 1012 may output adjusted BPE images 1044. Combining identification algorithm 1014 and adjustment algorithm 1016 into single hybrid algorithm 1012 may increase a computational efficiency of steps 302 and 304.

The identification algorithm may include a BPE algorithm 1018, trained to identify BPE. The examples where the ground truth includes BPE segmentation image 1008 is the second part of training pair 1006 may be used to train the BPE algorithm 1018 as indicated by dashed arrow 1017. The BPE algorithm 1018 may automatically identify ROIs of a contrast enhanced breast image as corresponding to BPE. Additionally, the examples where the second part of training pair 1006 ground truth includes suspicious area segmentation image 1010 may be used to train suspicious area algorithm 1020 as indicated by dashed arrow 1019. Suspicious area algorithm 1020 may automatically identify ROIs of a contrast enhanced breast image as corresponding to a suspicious area.

Once trained, acquired images 1042 may be input into identification algorithm 1014. Acquired images 1042 may be acquired contrast enhanced breast images, such as the contrast enhanced breast images acquired at step 301 of method 300. Together, trained BPE algorithm 1018 and trained suspicious area algorithm 1020 may automatically distinguish BPE from suspicious areas of acquired images 1042.

As one example, BPE algorithm 1018 and suspicious area algorithm 1020 may each utilize contrast levels to identify regions corresponding to BPE and regions corresponding to the suspicious area. A contrast enhancement due to BPE may be less than a contrast enhancement due to a suspicious area. In this way, a contrast threshold may be used to distinguish an ROI as corresponding to either BPE or a suspicious area. As an alternate example, BPE algorithm 1018 and suspicious area algorithm 1020 may each be machine learning algorithms, such as a deep learning algorithm. The machine learning algorithm may be an algorithm which performs segmentation tasks. As one example, the machine learning algorithm may be Unet, Vnet, or Mask-RCNN, among others. In a further example when the contrast enhanced breast images are acquired using CEM, the BPE algorithm 1018 and the suspicious area algorithm 1020 may each be based on acquisition time. During a CEM exam, multiple CEM low and high energy x-ray images pairs may be acquired over time (CC and MLO view for e.g). Contrast media may collect in a lesion or suspicious area before pooling in healthy breast tissue to cause BPE. For this reason, an algorithm may identify contrast enhanced ROIs in CEM images collected before a threshold time as ROIs corresponding to lesions or suspicious areas. Any additional ROIs of increased contrast appearing in recombined x-ray images acquired after the threshold time may then be identified at ROIs corresponding to BPE. More generally the BPE or suspicious lesion algorithms may be applied to multiple CEM images of various views acquired during an exam and timing information can be used as input.

Alternatively, suspicious area algorithm 1020 may be a machine learning algorithm, such as a deep learning algorithm. For example, the algorithm may include a you only look once (YOLO) architecture. Additionally or alternatively, suspicious area algorithm 1020 may utilize radiomics features extraction and classification, among other imaging processing tools to identify suspicious areas in the contrast enhanced breast image.

As an alternate embodiment of identification algorithm 1014, identification algorithm 1014 may include a combined BPE and suspicious area algorithm 1022. Both BPE segmentation image 1008 and suspicious area segmentation image 1010 may be used in combination as ground truth to train combined BPE and suspicious area algorithm 1022 as indicated by arrow 1023. Combined BPE and suspicious area algorithm 1022 may distinguish BPE and suspicious areas when input acquired images 1042. As one example, BPE and suspicious area algorithm 1022 may be a machine learning/deep learning algorithm that performs multi-instance segmentation, such as, but not limited to, Mask-RCNN. Combining BPE and suspicious area identification into a single multi-task algorithm may decrease an amount of human time demanded for training algorithms and may further increase a computational efficiency of identification algorithm 1014.

In some embodiments, an output of the trained suspicious area algorithm 1020 may be combined with BPE segmentation image 1008 for training BPE algorithm 1018 as indicated by a combination of arrow 1017 and arrow 1021. Said another way, suspicious area algorithm 1020 may be first trained using a ground truth including suspicious area segmentation 1010. Contrast enhanced images may then be input to suspicious area algorithm 1020 which outputs contrast images in which ROIs corresponding to suspicious areas are segmented. The outputs in which ROIs corresponding to suspicious areas are identified may be used in addition to BPE segmentation images 1008 to train BPE algorithm 1018. In this way, suspicious areas such as lesions or foreign objects as well as other artifacts, such as the nipple, which do not affect the BPE are already detected by the suspicious area algorithm 1020 and an accuracy of BPE algorithm 1018 may be increased. In one embodiment regions of the image that are above a certain threshold but not a suspicious area may considered as BPE.

Adjustment algorithm 1016 may be configured to receive outputs of identification algorithm 1014 and automatically adjust a display of BPE. Adjustment algorithm 1016 may be an image processing algorithm with instructions for blending pixels or fusing colors of pixels according to a desired output. In alternate examples, the adjustment algorithm may be a deep learning network configured to highlight areas and/or features of an image.

Adjustment algorithm 1016 may include a decrease contrast algorithm 1028. Decrease contrast algorithm 1028 may be configured to decrease a contrast of the ROI identified as corresponding to BPE. As one example, the contrast may be decreased to be equal to a background contrast level. In an alternate embodiment, adjustment algorithm 1016 may include an increase contrast algorithm 1030. Increase contrast algorithm 1030 may be configured to increase a contrast of the ROI identified as corresponding to BPE. As one example, the contrast may be increased to be equal to or greater than a contrast of an ROI identified as corresponding to a suspicious area. In an alternate embodiment, adjustment algorithm 1016 may include a highlight area algorithm 1032. Highlight area algorithm 1032 may be configured to overlay a partially transparent colored image corresponding to the ROI identified as BPE over the contrast enhanced image. The partially transparent colored image may aid the clinician in differentiating the BPE from the rest of the image.

In one embodiment, an output of BPE algorithm 1018 may be an input of adjustment algorithm 1016 as shown by arrow 1024. The output of BPE algorithm 1018 fed into adjustment algorithm 1016 may be based on training BPE segmentation image 1008 (arrow 1017) or may be based on training with suspicious area algorithm 1020 (arrow 1021) followed by training with BPE segmentation image 1008 (arrow 1017). In an alternate embodiment, an output BPE algorithm 1018 may be input to adjustment algorithm 1016 in combination with an output of suspicious area algorithm 1020 (following arrow 1025). BPE algorithm 1018 may be combined with suspicious area algorithm 1020 sequentially (e.g., suspicious area algorithm 1020 followed by BPE algorithm 1018) or simultaneously.

In a further embodiment, an output of BPE and suspicious area algorithm 1022 may be an input of adjustment algorithm 1016 (following arrow 1026). Adjusted BPE images 1044 may be output after a sequential iteration of identification algorithm 1014 and adjustment algorithm 1016 and/or as an output of single hybrid algorithm 1012.

Examples of possible displays of adjusted BPE images 1044 are shown in FIGS. 6-8. The displayed images may include a morphological image (e.g., breast image without contrast enhancement), a contrast enhanced breast image (e.g., contrast enhanced image), and a corresponding contrast enhanced breast image with adjusted BPE (e.g., contrast enhanced adjusted BPE image). In some examples, the images may be acquired by a CEM exam and the morphological image may include a low energy image and the contrast enhanced image may be a recombined contrast enhanced image and the contrast enhanced image with adjusted BPE may be an adjusted recombined image.

FIG. 6 shows a first example 600 of a display 602 showing a morphological image 604, a contrast enhanced with BPE 606, and contrast enhanced adjusted BPE image 608. Contrast enhanced image with BPE 606 may be an unadjusted contrast enhanced breast image. Contrast enhanced adjusted BPE image 608 may be an output by an adjustment algorithm (such as adjustment algorithm 1016 of FIG. 10). In some examples, contrast enhanced adjusted BPE image 608 may be a BPE free image. In alternate examples, contrast enhanced adjusted BPE image 608 may either enhance a contrast of BPE or highlight the BPE region. As one example, display 602 may be a large display, such as a full area of a computer monitor. Example 600 may show morphological image 604 at a left most portion of display 602. Contrast enhanced image with BPE 606 may be placed to the right of morphological image 604. Contrast enhanced adjusted BPE image 608 may be positioned to the right of contrast enhanced image with BPE 606. Other arrangements of morphological image 604, contrast enhanced image with BPE 606 and contrast enhanced adjusted BPE image 608 wherein each is fully visible at the same time on display 602 have also been considered. In some examples, first example 600 may be a first type of display, automatically selected for a large format display.

FIG. 7 shows a second example 700 of a display 702 which may in a first instance 710 show morphological image 704. In a second instance 712, display 702 may show contrast enhanced with BPE image 706. A fade effect where a transparency of morphological image 704 is gradually increased while at the same time decreasing a transparency of contrast enhanced with BPE image 706 in the same screen area may be used in transition from first instance 710 to second instance 712. In a third instance 714, display 702 may show contrast enhanced adjusted BPE image 708. In some examples, contrast enhanced adjusted BPE image 708 may be a BPE free image. In alternate examples, contrast enhanced adjusted BPE image 708 may include enhanced or highlighted BPE. Similarly, the fade effect may be used to transition between second instance 712 and third instance 714. In this way, morphological image 704, contrast enhanced with BPE image 706 and contrast enhance adjusted BPE image 708 may be shown sequentially. In some examples, an order of first instance 710, second instance 712, and third instance 714 may be reversed or swapped. Sequential viewing may be desired when a display such as display 702 has a reduced area, such as a minimized window, and/or a mobile device and a multi-image view as shown in first example 600 would make images too small, thereby obscuring details which are demanded for the clinician to perform an analysis. Additionally, the fade effect may overlay the images which may allow for easier comparison and analysis by the clinician.

In some examples a displayed image (e.g., morphological image 704, contrast enhanced with BPE image 706, and/or contrast enhanced adjusted BPE image 708) may be a height 718 and width 720 and display 702 may be a height 722 and width 724. A ratio of height and width may be referred to as a form factor. A form factor of the displayed image may be dependent on settings of an imaging device (e.g., digital mammography system 100 of FIG. 1) and may be a fixed value. In other words, adjustment algorithm 1016 may not adjust a form factor of the BPE image. Adjusting a form factor of the displayed image may lead to undesirable distortion of the displayed image. For this reason, a form factor of the displayed image may be kept constant and may not be adjusted to match a form factor of the display. For example, a form factor of a display may change if the display is rotated, and the form factor displayed image may not change. Displaying the image sequentially as shown in FIG. 7 may be demanded to maintain the form factor of the displayed image while also maintaining a large enough displayed image for the clinician to make an accurate diagnosis.

FIG. 8 shows a third example 800 of a display 802 which may in a first instance 812 show morphological image 804. In a second instance 814, display 802 may show contrast enhanced with BPE image 806. In a third instance 816, display 802 may show a contrast enhanced image with a first level of BPE 808. In a fourth instance 818, display 802 may show a contrast enhanced image with a second level of BPE 810. In some examples, a difference in contrast between the BPE region and background in first contrast enhanced level of BPE image 808 may be different than the difference in contrast between the BPE region and the background in second contrast enhanced level of BPE image 810. Said another way, a contrast of BPE in image 808 may be different than a contrast of BPE in image 810. In some examples a contrast of the first level of BPE 808 may be greater than second level of BPE 810. In alternate examples the contrast of BPE in image 808 may be less than second level of BPE 810. In alternate examples contrast enhanced first level BPE 808 may be highlighted in an overlay of a first transparency and second level of BPE may include the same overly with a second transparency, the first transparency greater than the second transparency. In some examples, image with second reduced level of BPE 810 may be a BPE free image. In some examples, third example 800 may include additional instances with levels of BPE between the first reduced level and the second reduced level. The clinician may transition from displaying the first instance 812 and subsequent instances (e.g., second instance 814, third instance 816, and so on) by scrolling. Scrolling may include transitions between the instances wherein the first instance is moved off of display 802 in a first direction while simultaneously the following instance is moved onto the screen from a second direction. In this way the clinician may view morphological image 804, recombined with BPE image 806 and a plurality of adjusted BPE images, including, for example first BPE level image 808 and second BPE level image 810 in a sequential fashion. In some examples, transitions between instance (e.g., first instance 812 and second instance 814) may be fading transitions as described above with respect to FIG. 7, or an instance may be instantaneously replaced by a subsequent instance without a fading transition or scrolling transition.

The technical effect of method 300 is to automatically generate contrast enhanced breast images with the presentation of BPE adjusted to be different from the presentation of a suspicious area such as a lesion. In some examples, the BPE may be removed from the contrast enhanced image or in alternate examples, the BPE may be highlighted. BPE corresponds to healthy tissue, and automatically reducing or removing BPE may allow a clinician to accurately and precisely determine an existence and location of a possibly cancerous legion. Further, the method of displaying the relevant images to the clinician may be adjusted to be adapted to a minimized window or a mobile device display. In this way, efficiency and accuracy of screening contrast enhance breast images may be increased.

The disclosure also provides support for a method, comprising: automatically distinguishing a first region of interest (ROI) of a contrast enhanced breast image from a second ROI of the contrast enhanced breast image, wherein the first ROI corresponds to background parenchymal enhancement (BPE) and the second ROI corresponds to a suspicious area, automatically adjusting an appearance of the first ROI and generating an adjusted BPE image, and displaying the contrast enhanced breast image and the adjusted BPE image at a display. In a first example of the method, automatically distinguishing the first ROI from the second ROI include inputting the contrast enhanced breast image to a BPE algorithm trained using image pairs and a BPE segmentation image. In a second example of the method, optionally including the first example, the method further comprises training a suspicious area algorithm using image pairs and a suspicious area segmentation image and after training the suspicious area algorithm, training a BPE algorithm using an output of the suspicious area algorithm, the trained BPE algorithm used for automatically distinguishing the first ROI from the second ROI. In a third example of the method, optionally including one or both of the first and second examples, automatically distinguishing the first ROI from the second ROI and automatically adjusting the appearance includes inputting the contrast enhanced breast image to a single hybrid algorithm. In a fourth example of the method, optionally including one or more or each of the first through third examples, displaying the contrast enhanced breast image and adjusted BPE image includes displaying the contrast enhanced breast image and the adjusted BPE image simultaneously. In a fifth example of the method, optionally including one or more or each of the first through fourth examples, displaying the contrast enhanced breast image and adjusted BPE image includes displaying the contrast enhanced breast image and the adjusted BPE image sequentially. In a sixth example of the method, optionally including one or more or each of the first through fifth examples, the display is a mobile device. In a seventh example of the method, optionally including one or more or each of the first through sixth examples, the method further comprises, after displaying the contrast enhanced breast image and adjusted BPE image, fading or scrolling between an instance showing the contrast enhanced breast image and an instance showing the adjusted BPE image. In a eighth example of the method, optionally including one or more or each of the first through seventh examples, contrast enhanced breast image is a recombined image acquired by contrast enhanced mammography exam.

The disclosure also provides support for an image processing system, comprising: a display device, an image processing device communicatively coupled to the display device and including a processor and non-transitory memory storing instructions executable by the processor to: distinguish a region of interest (ROI) of a contrast enhanced breast image corresponding to background parenchymal enhancement (BPE) using a BPE algorithm that has been trained using image pairs including a ground truth BPE segmented image and a suspicious area algorithm that has been trained using image pairs including a ground truth suspicious area segmented image, adjust, using an adjustment algorithm, the ROI of the contrast enhanced breast image corresponding to BPE, and output the adjusted contrast enhanced breast image to the display device. In a first example of the system, the instructions to adjust the ROI of the contrast enhanced breast image corresponding to BPE further include to increase a contrast of the ROI of the contrast enhanced breast image corresponding to BPE, decrease the contrast of the ROI of the contrast enhanced breast image corresponding to BPE, or highlight the ROI of the contrast enhanced breast image corresponding to BPE. In a second example of the system, optionally including the first example, the instructions further include to output an unadjusted contrast enhanced breast image and a corresponding contrast enhanced breast image without enhanced contrast. In a third example of the system, optionally including one or both of the first and second examples, the instructions to adjust the ROI of the contrast enhanced breast image include to adjust the ROI of the contrast enhanced breast image to a first level and to adjust the ROI of the contrast enhanced breast image to a second level. In a fourth example of the system, optionally including one or more or each of the first through third examples, the instructions include to output the contrast enhanced breast image adjusted to the first level in sequence with the contrast enhanced breast image adjusted to the second level. In a fifth example of the system, optionally including one or more or each of the first through fourth examples, the BPE algorithm and the suspicious area algorithm are machine learning algorithms. In a sixth example of the system, optionally including one or more or each of the first through fifth examples, the BPE algorithm and the suspicious area algorithm are included in a combined BPE and suspicious area algorithm. In a seventh example of the system, optionally including one or more or each of the first through sixth examples, an output of the BPE algorithm and an output of suspicious area algorithm are sequential or simultaneous inputs of an adjustment algorithm.

The disclosure also provides support for a system for digital mammography, comprising: an x-ray source in communication with a detector, a display device, and a computing device in communication with the display device and with the detector, the computing device including a processor and non-transitory memory storing instructions executable by the processor to: acquire a low energy x-ray image and a high energy x-ray image, output a recombined image based on the low energy x-ray image and the high energy x-ray image, and identify background parenchymal enhancement (BPE) in the recombined image and adjust recombined image, and output an adjusted recombined image to the display device. In a first example of the system, the instructions to identify BPE and adjust the recombined image are included in a single hybrid algorithm. In a second example of the system, optionally including the first example, the instructions to identify BPE in the recombined image are based on a contrast threshold.

As used herein, an element or step recited in the singular and preceded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A method, comprising:
automatically distinguishing a first region of interest (ROI) of a contrast enhanced breast image, wherein the first ROI corresponds to background parenchymal enhancement (BPE);
automatically adjusting an appearance of the first ROI and generating an adjusted BPE image; and
displaying the contrast enhanced breast image and the adjusted BPE image at a display.

2. The method of claim 1, further comprising automatically distinguishing a second ROI of the contrast enhanced breast image, wherein the second ROI corresponds to a suspicious area, and automatically distinguishing the first ROI from the second ROI, including inputting the contrast enhanced breast image to a BPE algorithm trained using a contrast enhanced breast image and a BPE segmentation image.

3. The method of claim 2, wherein the method further comprises training a suspicious area algorithm using a contrast enhanced breast image and a suspicious area segmentation image and after training the suspicious area algorithm, training a BPE algorithm using an output of the suspicious area algorithm, the trained BPE algorithm used for automatically distinguishing the first ROI from the second ROI.

4. The method of claim 2, wherein automatically distinguishing the first ROI from the second ROI and automatically adjusting the appearance includes inputting the contrast enhanced breast image to a single hybrid algorithm.

5. The method of claim 1, wherein displaying the contrast enhanced breast image and adjusted BPE image includes displaying the contrast enhanced breast image and the adjusted BPE image simultaneously.

6. The method of claim 1, wherein displaying the contrast enhanced breast image and adjusted BPE image includes displaying the contrast enhanced breast image and the adjusted BPE image sequentially.

7. The method of claim 6, wherein the display is a mobile device.

8. The method of claim 6, wherein the method further comprises, after displaying the contrast enhanced breast image and adjusted BPE image, fading or scrolling between an instance showing the contrast enhanced breast image and an instance showing the adjusted BPE image.

9. The method of claim 1, wherein contrast enhanced breast image is a recombined image acquired by contrast enhanced mammography exam.

10. An image processing system, comprising:
a display device;
an image processing device communicatively coupled to the display device and including a processor and non-transitory memory storing instructions executable by the processor to:
distinguish a region of interest (ROI) of a contrast enhanced breast image corresponding to background parenchymal enhancement (BPE) using a BPE algorithm that has been trained using image pairs including a ground truth BPE segmented image and a suspicious area algorithm that has been trained using image pairs including a ground truth suspicious area segmented image;
adjust, using an adjustment algorithm, the ROI of the contrast enhanced breast image corresponding to BPE; and
output the adjusted contrast enhanced breast image to the display device.

11. The image processing system of claim 10, wherein the instructions to adjust the ROI of the contrast enhanced breast image corresponding to BPE further include to increase a contrast of the ROI of the contrast enhanced breast image corresponding to BPE, decrease the contrast of the ROI of the contrast enhanced breast image corresponding to BPE, or highlight the ROI of the contrast enhanced breast image corresponding to BPE.

12. The image processing system of claim 10, wherein the instructions further include to output an unadjusted contrast enhanced breast image and a corresponding contrast enhanced breast image without enhanced contrast.

13. The image processing system of claim 10, wherein the instructions to adjust the ROI of the contrast enhanced breast image corresponding to BPE include to adjust the ROI of the contrast enhanced breast image to a first level and to adjust the ROI of the contrast enhanced breast image to a second level.

14. The image processing system of claim 13, wherein the instructions include to output the contrast enhanced breast image adjusted to the first level in sequence with the contrast enhanced breast image adjusted to the second level.

15. The image processing system of claim 10, wherein the BPE algorithm and the suspicious area algorithm are machine learning algorithms.

16. The image processing system of claim 10, wherein the BPE algorithm and the suspicious area algorithm are included in a combined BPE and suspicious area algorithm.

17. The image processing system of claim 10, wherein an output of the BPE algorithm and an output of suspicious area algorithm are sequential or simultaneous inputs of an adjustment algorithm.

18. A system for digital mammography, comprising:
an x-ray source in communication with a detector;
a display device; and
a computing device in communication with the display device and with the detector, the computing device including a processor and non-transitory memory storing instructions executable by the processor to:
acquire a low energy x-ray image and a high energy x-ray image;
output a recombined image based on the low energy x-ray image and the high energy x-ray image; and
identify background parenchymal enhancement (BPE) in the recombined image and adjust recombined image; and
output an adjusted recombined image to the display device.

19. The system of claim 18, wherein the instructions to identify BPE and adjust the recombined image are included in a single hybrid algorithm.

20. The system of claim 18, wherein the instructions to identify BPE in the recombined image are based on a contrast threshold.
